(19) United States
(12) Patent Application Publication
(10) Pub. No.: US 2006/0004102 A1
(43) Pub. Date: Jan. 5, 2006

(54) Polymorphic forms of nateglinide

(76) Inventors: Shlomit Wizel, Petah Tiqva (IL); Gustavo Frenkel, Beer Sheva (IL); Boaz Gome, Rishon-Lezion (IL)

Correspondence Address: KENYON & KENYON, ONE BROADWAY, NEW YORK, NY, 10004, US

(21) Appl. No.: 11/126,050

(22) Filed: May 9, 2005

Related U.S. Application Data
(60) Provisional application No. 60/569,047, filed on May 7, 2004.

(51) Int. Cl. A61K 31/198

(52) U.S. Cl. 514/563; 562/450

(57) ABSTRACT

Provided are crystalline forms of nateglinide and processes for their preparation.

### CROSS-REFERENCE TO RELATED APPLICATIONS

This application claims the benefit of U.S. Provisional Application Ser. No. 60/569,047 filed May 7, 2005, the disclosure of which is incorporated by reference in its entirety herein.

### FIELD OF THE INVENTION

The present invention relates to the solid state chemistry of nateglinide.

### BACKGROUND OF THE INVENTION

Nateglinide, known as (−)-N-(trans-4-isoporpylcyclohexanecarbonyl)-D-Phenylalanine, has the following structure and characteristics: 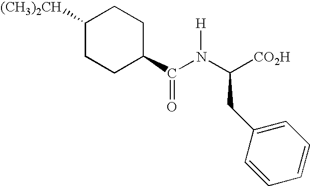
Formula C₁₉H₂₇NO₃
Molecular Weight 317.42
Exact Mass 317.199093
Composition C 71.89% H 8.57% N 4.41% O 15.12%

Nateglinide is marketed as STARLIX, which is prescribed as oral tablets having a dosage of 60 mg and 120 mg for the treatment of type II diabetes. STARLIX may be used as monotherapy or in combination with metaformin to stimulate the pancreas to secrete insulin. According to the maker of STARLIX, nateglinide is a white powder that is freely soluble in methanol, ethanol, and chloroform, soluble in ether, sparingly soluble in acetonitrile and octanol, and practically insoluble in water.

The present invention relates to the solid state physical properties of nateglinide. These properties may be influenced by controlling the conditions under which nateglinide is obtained in solid Form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state Form of a compound may also affect its behavior on compaction and its storage stability.

These practical physical characteristics are influenced by the conformation and orientation of molecules in the unit cell, which defines a particular polymorphic Form of a substance. The polymorphic Form may give rise to thermal behavior different from that of the amorphous material or another polymorphic Form. Thermal behavior is measured in the laboratory by such techniques as capillary melting point, thermogravimetric analysis (TGA) and differential scanning calorimetry (DSC) and may be used to distinguish some polymorphic forms from others. A particular polymorphic Form may also give rise to distinct spectroscopic properties that may be detectable by powder X-ray crystallography, solid state C NMR spectrometry and infrared spectrometry.

Nateglinide exists in various crystalline forms. U.S. Pat. Nos. 5,463,116 and 5,488,150 disclose two crystal forms of nateglinide, designated B-type and H-type, and processes for their preparation. These patents are incorporated herein by reference for their disclosure of the forms. Both forms are characterized by melting point, XRPD pattern, IR spectrum in KBr and DSC thermogram. According to these patents, B-type has a melting point of 129-130° C. while H-type has a melting point of 136-142° C. The H-type crystals are characterized in these patents by a powder XRD pattern with peaks at 8.1, 13.1, 19.6 and 19.9±0.2 degrees 2θ, and a strong reflection between 15 and 17±0.2 degrees 2θ. The B-type crystal is reported to lack these peaks and have a weak reflection between 15 and 17±0.2 degrees 2θ. H-type crystals are reported to have an IR spectrum with characteristic absorptions at about 1714, 1649, 1542 and 1214 cm⁻¹. These absorptions are reported to be missing in the spectrum of B-type crystals.

According to U.S. Pat. No. 5,463,116, B-type crystals are unstable and susceptible to change during grinding as demonstrated by DSC. The DSC thermogram of B-type shows a sharp endotherm at 131.4° C. before grinding while that of H-type shows a sharp endotherm at 140.3° C. After grinding, the DSC thermogram of B-type shows a second endotherm at 138.2° C., suggesting a solid-solid transformation during grinding.

According to U.S. Pat. No. 5,463,116, the temperature during crystallization and filtration determines whether the crystal Form is B-type or H-type. Temperatures above 10° C., more preferably above 15° C., lead to formation of H-type, while those below 10° C. lead to formation of B-type.

Another crystalline form of nateglinide designated Type-S is disclosed in two Chinese articles: ACTA Pharm. Sinica 2001, 36(7), 532-34 and Yaowu Fenxi Zazhi, 2001, 21(5), 342-44. Form S is reported to be distinguisheable from Forms B and H by a melting point of 172.0 C, a Fourier Transform IR with a peak at 3283 cm⁻¹ (as supposed to 3257 cm⁻¹, and 3306 cm⁻¹ for Forms B and H respectively) and an XRPD pattern with a strong peak at 3.78±0.2 degrees 2θ.

WO03076393 discloses salts of nateglinide.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. New polymorphic forms of nateglinide has now been discovered.

### SUMMARY OF THE INVENTION

In one aspect, the present invention provides a crystalline form of nateglinide ammonium salt (Phi) characterized by a powder XRD pattern with peaks at 4.2, 4.9, 12.7, 13.4, 14.8, 15.8, 17.5, 19.3±0.2 degrees 2θ.

In one aspect, the present invention provides a process for preparing the above crystalline form comprising precipitating the crystalline form from a mixture of water and methanol under basic conditions in presence of ammonia, and recovering the crystalline form.

In one embodiment, this process comprises:
a) preparing an acidic mixture of nateglinide in a mixture of water and methanol;
b) combining the mixture with a base and a source of ammonium ions to obtain a precipitate; and
c) recovering the nateglinide ammonium salt crystalline form.

In another embodiment, this process comprises:
a) preparing an heterogeneous mixture of nateglinide in a mixture of water, methanol, a base and a source of ammonium ions;
b) precipitating the crystalline form from the mixture; and
c) recovering the crystalline form.

In another aspect, the present invention provides a crystalline form of nateglinide (Form Lambda) characterized by a powder XRD pattern with peaks at 3.9, 4.8, 8.8, 14.5, 17.8, 19.1, 20.0±0.2 degrees 2θ.

In another aspect, the present invention provides a process of preparing the above crystalline form, comprising crystallizing the crystalline form from a mixture of nateglinide in a mixture of water and acetone.

Also provided are pharmaceutical compositions and methods of lowering blood glucose level in a mammal in need thereof with administration of the pharmaceutical compositions.

### BRIEF DESCRIPTION OF THE FIGURE

FIG. 1 is an X-ray Powder Diffraction (XRPD) pattern of nateglinide ammonium Form Phi.

FIG. 2 is an X-ray Powder Diffraction (XRPD) pattern of nateglinide Form Lambda.

### DETAILED DESCRIPTION OF THE INVENTION

The present invention provides two crystal forms of nateglinide, designated form phi and lambda. The present invention continues the naming system of U.S. 2005/0014949, U.S. 2004/0181089, U.S. 2004/0116526 and U.S. 2005/0014836.

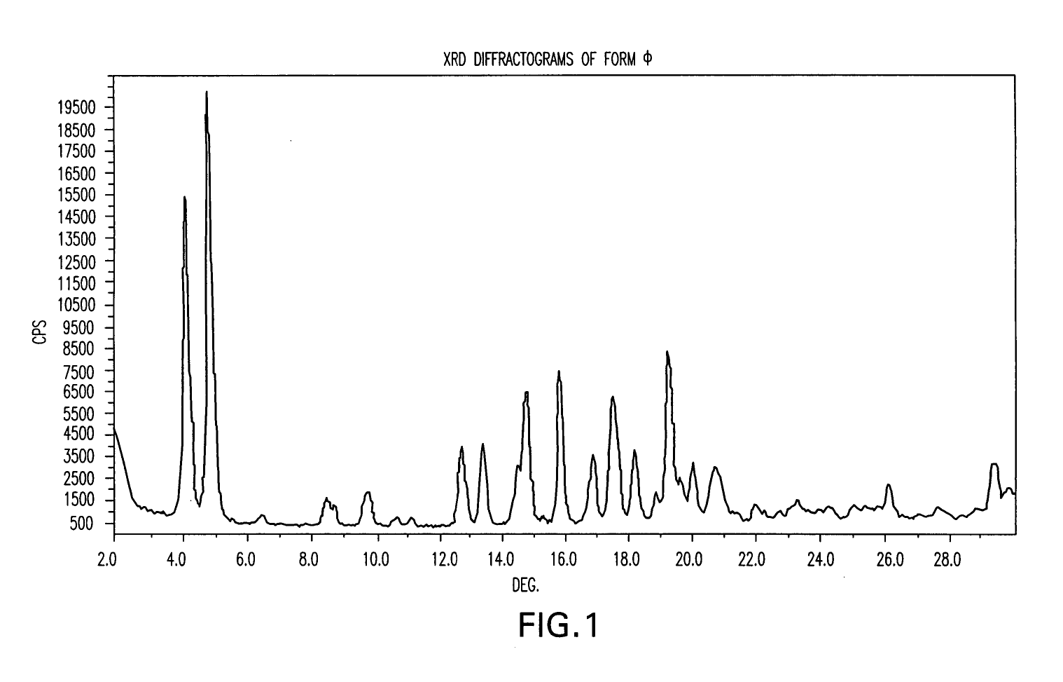

The present invention provides for a crystalline form of nateglinide ammonium salt (Phi) characterized by a powder XRD pattern with peaks at 4.2, 4.9, 12.7, 13.4, 14.8, 15.8, 17.5, 19.3±0.2 degrees 2θ. The actual powder XRD pattern is provided as FIG. 1. Form Phi may be prepared from a mixture of methanol and water in presence of ammonium ions. In addition for formulation, the ammonium salt may be used for purification of nateglinide.

In one embodiment, Form Phi is prepared by precipitation from an acidic mixture containing nateglinide, methanol and water. The mixture preferably contains from about a 1:1 to about a 4:1, more preferably about a 3:1 mixture of methanol and water (v/v). Nateglinide is freely soluble in methanol but insoluble in water. The ratio of water to methanol is preferably chosen as to allow for a solution. A preferred pH for the acidic mixture is about 4. The mixture of water and methanol may be heated to further increase solubility of the nateglinide in the mixture. A suitable temperature is about 30° C. to about 50° C. More preferably, the temperature is about 40° C.

The crystallization of Form Phi is carried out by basifying the acidic mixture of nateglinide until precipitation occurs. Preferably a basic reagent that serves both as a base and a source of ammoinum ions is used. The precipitation may be observed by cloudiness of the mixture or formation of a relatively few crystals. The mixture is preferably basified to a pH of about 5 or more. To further induce crystallization, the reaction mixture may be cooled, preferably to a temperature of about −10° C. to about 10° C. The crystals may be recovered by conventional techniques such as filtration.

In another embodiment, Form Phi may be prepared from a heterogeneous mixture of nateglinide in a mixture of methanol, water and a base. Preferably a basic reagent that serves both as a base and a source of ammoinum ions is used. Such basic reagent causes formation of the relatively insoluble ammonium salt, Form Phi. The pH is preferably about 5 or more. The ratio of methanol to water is preferably about 8 to about 1 vol/vol of methanol to water. Preferably, the mixture is stirred for a sufficient time. The resulting mixture may be heated, preferably to a temperature of about 30° C. to about 50° C. More preferably, the mixture may be heated to a temperature of about 40° C. The mixture may be cooled, preferably to a temperature of −10° C. to about 10° C., to increase the yield. The crystals may be recovered by conventional techniques such as filtration.

Basifying of the reaction mixture to a pH greater than 5 increases the precipitation.

The present invention also provides for a crystalline form of nateglinide (Form Lambda) characterized by an powder XRD pattern with peaks at 3.9, 4.8, 8.8, 14.5, 17.8, 19.1, 20.0±0.2 degrees 2. Appropiate powder XRD figure corresponds to figure no. 2. Form Lambda may be prepared by crystallization from a mixture of water and acetone. Preferably the mixture is about a 4:1 to about 1:1 acetone to water (vol/vol). A mixture of nategiline is prepared in the mixture of water and acetone. The mixture may be heated to aid in dissolution. In one embodiment, the mixture is heated to a temperature of about 30° C. to about 40° C. More preferably, the temperature is about 35° C.

In one embodiment, after dissolution, crystallization is induced by cooling the mixture. Preferably cooling is carried out at a temperature of about −10° C. to about 10° C. The crystals may be recovered by conventional techniques such as filtration.

The crystal forms obtained may be dried. Preferably drying is carried out at reduced pressure (below 1 atm), more preferably below about 100 mmHg.

The pH for the processes of the present invention may be adjusted with bases within the skill in the art. Examples of bases include, for example, an alkali metal hydroxide, an alkaline earth metal hydroxide, an alkali metal hydride, an alkali metal carbonate, alkaline earth metal carbonate, hydrogencarbonate, basic alumina and ammonium hydroxide. Specific examples of bases include: sodium hydroxide, potassium hydroxide, calcium hydroxide, magnesium hydroxide, sodium carbonate, potassium carbonate, sodium hydrogencarbonate, potassium hydrogencarbonate and calcium carbonate. When forming an ammonium salt, an ionic reagent forming both a base and providing a source of ammonium ions may be used.

The starting material used for the processes of the present invention may be any crystalline or amorphous form of nateglinide, including various solvates and hydrates. With crystallization processes, the crystalline form of the starting material does not usually affect the final result. One of skill in the art would appreciate the manipulation of the starting material within skill in the art to obtain a desirable form with trituration.

The processes of the present invention may also be practiced as the last step of prior art processes that synthesize nateglinide.

Many processes of the present invention involve crystallization out of a particular solvent. One skilled in the art would appreciate that the conditions concerning crystallization may be modified without affecting the form of the polymorph obtained. For example, when mixing nateglinide in a solvent to form a solution, warming of the mixture may be necessary to completely dissolve the starting material. If warming does not clarify the mixture, the mixture may be diluted or filtered. To filter, the hot mixture may be passed through paper, glass fiber or other membrane material, or a clarifying agent such as celite. Depending upon the equipment used and the concentration and temperature of the solution, the filtration apparatus may need to be preheated to avoid premature crystallization.

The conditions may also be changed to induce precipitation. A preferred way of inducing precipitation is to reduce the solubility of the solvent. The solubility of the solvent may be reduced, for example, by cooling the solvent.

In one embodiment, an anti-solvent is added to a solution to decrease its solubility for a particular compound, thus resulting in precipitation. Another way of accelerating crystallization is by seeding with a crystal of the product or scratching the inner surface of the crystallization vessel with a glass rod. Other times, crystallization may occur spontaneously without any inducement.

Nateglinide of defined particle size may be produced by known methods of particle size reduction starting with crystals, powder aggregates and course powder of the new crystalline forms of nateglinide. The principal operations of conventional size reduction are milling of a feedstock material and sorting of the milled material by size.

A fluid energy mill, or micronizer, is an especially preferred type of mill for its ability to produce particles of small size in a narrow size distribution. As those skilled in the art are aware, micronizers use the kinetic energy of collision between particles suspended in a rapidly moving fluid stream to cleave the particles. An air jet mill is a preferred fluid energy mill. The suspended particles are injected under pressure into a recirculating particle stream. Smaller particles are carried aloft inside the mill and swept into a vent connected to a particle size classifier such as a cyclone. The feedstock should first be milled to about 150 to 850 μm which may be done using a conventional ball, roller, or hammer mill. One of skill in the art would appreciate that some crystalline forms may undergo a transition to another form during particle size reduction.

Pharmaceutical compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups and suspensions. Suitable forms of parenteral administration include an aqueous or non-aqueous solution or emulsion, while for rectal administration suitable forms for administration include suppositories with hydrophilic or hydrophobic vehicle. For topical administration the invention provides suitable transdermal delivery systems known in the art, and for nasal delivery there are provided suitable aerosol delivery systems known in the art.

The pharmaceutical composition may contain only a single form of nateglinide, or a mixture of various forms of nateglinide, with or without amorphous form. In addition to the active ingredient(s), the pharmaceutical compositions of the present invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dixoide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, nateglinide and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The dosage and formulation of STARLIX may be used as a guidance. The dosage used is preferably from about 30 to about 240 mg of nateglinide, more preferably from about 60 to about 120 mg of nateglinide. The pharmaceutical compositions of the present invention, preferably in the form of a coated tablet, are administered from about 10 minutes to about 1 hours prior to a meal, more preferably about 15 minutes before each meal. The dose is not taken if the meal is skipped. The pharmaceutical compositions may also be used in combination with metaformin.

X-Ray Powder Diffraction:

X-Ray diffraction was performed on X-Ray powder diffractometer, Scintag, variable goniometer, Cu-tube, solid state detector. Sample holder: A round standard aluminum sample holder with round zero background quartz plate. The sample was put on the sample holder and immediately analyzed as is. Scanning parameters: Range: 2-40 deg 2θ, Continuos Scan, Rate: 3 deg./min.

### EXAMPLES

1. Preparation of Form φ (Phi)

A mixture of methanol (280 ml) and water (120 ml) was heated to 39° C. Nateglinide (20 grams) was added and stirred for 30 minutes to dissolution at pH=4. 5 grams of a 24% ammonium hydroxide solution were dropped to the mixture until pH=5 was reached. Small particles appeared at this point. The mixture was cooled to 0° C. during 5 hours, stirred at this temperature for 1 hour, and then filtered under vacuum. 30.69 grams of wet nateglinide were obtained. The wet product was dried under vacuum at 90° C. overnight (˜12 hours). 12.4 grams of dry nateglinide were obtained.

2. Preparation of Form φ (Phi)

A mixture of methanol (280 ml) and water (60 ml) and 4 grams of a 24% NH₄OH solution were heated to 40° C. 20 grams of nateglinide were added and stirred for 30 minutes but dissolution did not occur. The mixture was cooled to 0° C. during 5 hours, stirred at this temperature for 1 hour and then filtered under vacuum. 16.41 grams of wet nateglinide were obtained. The wet product was dried under vacuum overnight (˜12 hours). 7.54 gr of dry nateglinide were obtained.

3. Preparation of Form λ (Lambda)

A mixture of 4050 ml of acetone, 2700 ml of water and 450 gr of nateglinide were mixed and heated to 35° C. Dissolution at this temperature was almost complete. The mixture was filtered to remove insoluble matter. The solution was cooled to 20° C. The solution was cooled to −10° C. during 10 hours. Precipitation occurred at 8° C. The mixture was stirred at −10° C. for 3 hours and then filtered under vacuum. 747.4 gr of wet nateglinide were obtained. The wet product was dried under vacuum overnight (˜12 hours). 392.4 gr of dry nateglinide form λ were obtained.

Having thus described the invention with reference to particular preferred embodiments and illustrative examples, those in the art may appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Polymorphism in Pharmaceutical Solids, Drugs and the Pharmaceutical Sciences, Volume 95 may be used as a guidance. All references mentioned herein are incorporated in their entirety.

What is claimed is:

1. A crystalline form of nateglinide ammonium salt (Phi) characterized by a powder XRD pattern with peaks at 4.2, 4.9, 12.7, 13.4, 14.8, 15.8, 17.5, 19.3±0.2 degrees 2θ.

2. The crystalline form of claim 1, characterized by a powder XRD pattern as substantially depicted in FIG. 1.

3. A process for preparing the crystalline form of claim 1 comprising precipitating the crystalline form from a mixture of water and methanol under basic conditions in presence of ammonia, and recovering the crystalline form.

4. The process of claim 3, wherein the process comprises:
d) preparing an acidic mixture of nateglinide in a mixture of water and methanol;
e) combining the mixture with a base and a source of ammonium ions to obtain a precipitate; and
f) recovering the nateglinide ammonium salt crystalline form.

5. The process of claim 4, wherein the base is selected from the group consisting of: an alkali metal hydroxide, an alkaline earth metal hydroxide, an alkali metal hydride, an alkali metal carbonate, alkaline earth metal carbonate, hydrogencarbonate, basic alumina and ammonium hydroxide.

6. The process of claim 4, wherein the base and source of ammonium ions is ammonium hydroxide.

7. The process of claim 4, wherein the mixture in step a) is heated to a temperature of about 30° C. to about 50° C.

8. The process of claim 4, wherein the process further comprising a cooling step prior to step c).

9. The process of claim 8, wherein the cooling is performed to a temperature of about −10° C. to about 10° C.

10. The process of claim 4, wherein the methanol to water ratio is about 1:1 to about 4:1 (vol/vol).

11. The process of claim 4, wherein the pH of the acidic mixture is about 4.

12. The process of claim 4, wherein the pH in step b) is about 5 or more.

13. The process of claim 3, wherein the process comprises:
d) preparing an heterogeneous mixture of nateglinide in a mixture of water, methanol, a base and a source of ammonium ions;
e) precipitating the crystalline form from the mixture; and
f) recovering the crystalline form.

14. The process of claim 13, wherein the ratio of methanol to water is about 8 to about 1 vol/vol of methanol to water.

15. The process of claim 13, wherein the base is selected from the group consisting of:
an alkali metal hydroxide, an alkaline earth metal hydroxide, an alkali metal hydride, an alkali metal carbonate, alkaline earth metal carbonate, hydrogencarbonate, basic alumina and ammonium hydroxide.

16. The process of claim 13, wherein the base and source of ammonium ions is ammonium hydroxide.

17. The process of claim 13, wherein the mixture in step a) is heated to a temperature of about 30° C. to about 50° C.

18. The process of claim 13, wherein the process further comprising a cooling step prior to step c).

19. The process of claim 18, wherein the cooling is performed to a temperature of about −10° C. to about 10° C.

20. A pharmaceutical composition comprising crystalline nateglinide of claim 1 and a pharmaceutically acceptable excipient.

21. A method of lowering blood glucose level in a mammal comprising administering the pharmaceutical composition of claim 20 to the mammal in need thereof.

22. A crystalline form of nateglinide (Form Lambda) characterized by a powder XRD pattern with peaks at 3.9, 4.8, 8.8, 14.5, 17.8, 19.1, 20.0±0.2 degrees 2θ.

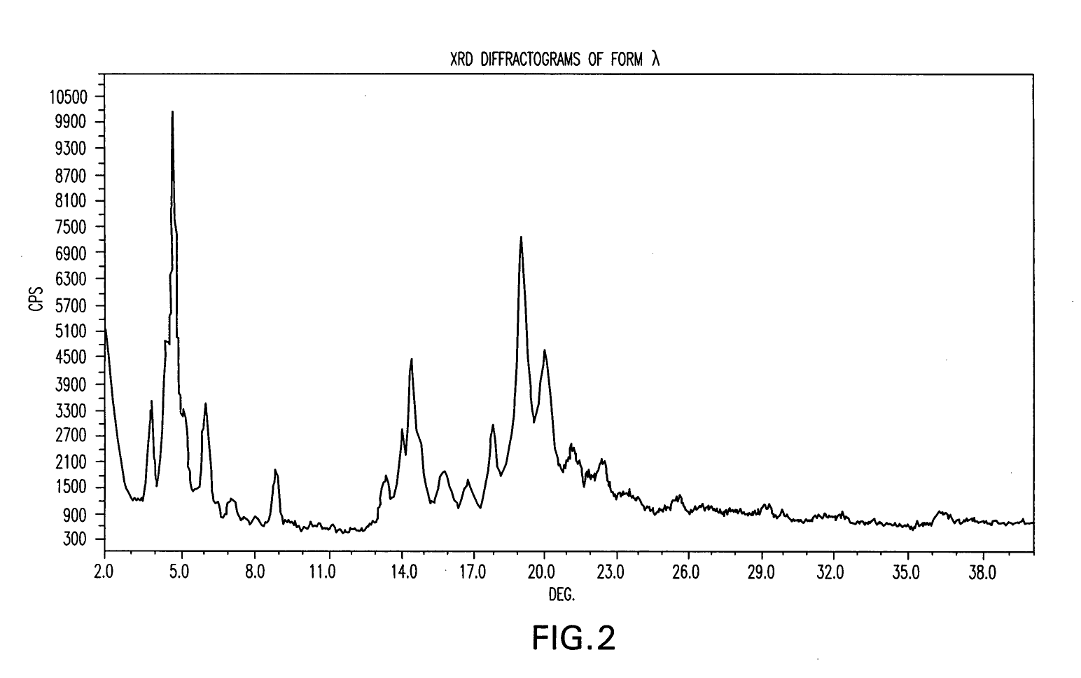

23. The crystalline form of claim 22, wherein the crystalline form is characterized by a powder XRD pattern as substantially depicted in FIG. 2.

24. A process of preparing the crystalline form of claim 23, comprising crystallizing the crystalline form from a mixture of nateglinide in a mixture of water and acetone.

25. The process of claim 24, wherein the mixture is about a 4:1 to about 1:1 acetone to water (vol/vol).

26. The process of claim 25, wherein crystallization is induced by cooling the mixture.

27. The process of claim 26, wherein cooling is carried out to a temperature of about −10° C. to about 10° C.

28. A pharmaceutical composition comprising crystalline nateglinide of claim 22 and a pharmaceutically acceptable excipient.

29. A method of lowering blood glucose level in a mammal comprising administering the pharmaceutical composition of claim 28 to a mammal in need thereof.
